(19) United States
(12) Patent Application Publication
(10) Pub. No.: US 2005/0273015 A1
(43) Pub. Date: Dec. 8, 2005

(54) Heart-activity monitoring with low-pressure, high-mass anatomy sensor contact

(75) Inventors: Peter T. Bauer, West Linn, OR (US); Peter M. Galen, Portland, OR (US); Martin Baumer, Carlton, OR (US)

Correspondence Address: ROBERT D. VARITZ, P.C., 4915 S.E. 33RD PLACE, PORTLAND, OR, 97202, US

(73) Assignee: Inovise Medical, Inc.

(21) Appl. No.: 11/200,753

(22) Filed: Aug. 9, 2005

Related U.S. Application Data
(63) Continuation-in-part of application No. 10/389,402, filed on Mar. 14, 2003.
(63) Continuation-in-part of application No. 10/389,530, filed on Mar. 14, 2003.
(60) Provisional application No. 60/600,269, filed on Aug. 10, 2004.
(60) Provisional application No. 60/364,768, filed on Mar. 14, 2002.
(60) Provisional application No. 60/364,770, filed on Mar. 14, 2002.
(60) Provisional application No. 60/364,405, filed on Mar. 14, 2002.

(51) Int. Cl. A61B 5/02; A61B 7/00

(52) U.S. Cl. 600/528

(57) ABSTRACT

An anatomy-contact sensor and related methodology for collecting at least heart-sound data. The sensor includes (a) a sensor body with an internal acoustic chamber having a mouth which is placeable adjacent, and preferably in contact with, a subject's anatomy, (b) a deflectible, preferably anatomy-contacting, preferably gas-permeable membrane spanning this mouth, and effectively sealing the chamber against through-passage in the mouth of all essentially but gas, (c) an acoustic-to-electrical-signal transducer mounted on the body and exposed to acoustic events occurring in the chamber, and (d) vacuumizing structure which is effective to hold the body in gripping contact with a subject's anatomy. From a methodological point of view, the invention involves the steps of (a) drawing a gas-permeable-membrane-sided acoustic chamber against, or adjacent, the anatomy of a subject utilizing a vacuum condition, and (b) following such drawing, collecting from the chamber anatomy-generated acoustic information which is based upon anatomy-induced acoustic events occurring in the chamber.

### CROSS REFERENCE TO RELATED APPLICATIONS

This application claims the priority filing date of U.S. Provisional Patent Application Ser. No. 60/600,269, filed Aug. 10, 2004 for Heart-Activity Monitoring With Multi-Axial, and Active Sound, Audio Detection. It is also (a) a CIP of Ser. No. 10/389,402, filed Mar. 14, 2003, for Method and Apparatus for Detecting and Transmitting Electrical and Related Audio Signals From a Single, Common Anatomical Site, which application claims priority from U.S. Provisional Patent Application Ser. No. 60/364,768, filed Mar. 14, 2002, for Reusable ECG/Sound Sensor, and (b) a CIP of Ser. No. 10/389,530, filed Mar. 14, 2003, for Audio/ECG Sensor Coupler With Integrated Signal Processing, which application claims priority from U.S. Provisional Patent Application Ser. No. 60/364,770, filed Mar. 14, 2002 for ECG/Sound Algorithm Adapter, and from U.S. Provisional Patent Application Ser. No. 60/364,405, filed Mar. 14, 2002 for Combined ECG/Sound assessment-Call Algorithm.

### BACKGROUND AND SUMMARY OF THE INVENTION

This invention relates to structure and methodology associated with the gathering, for review and study, of heart-produced sound (acoustic) and of ECG (electrical) signals/information. Principal features of the invention focus attention particularly on the gathering of acoustic information.

Practitioners in the field of cardiology have come to recognize the high-level importance of knowing about heart-produced acoustic signals because of the ability of such signals to enhance an understanding about a subject's heart condition.

The present invention features a unique sensor structure which is specially designed to become associated/related/attached to a subject's anatomy in a manner which allows for very accurate and confident gathering, especially, of heart-sound signals.

According to a preferred embodiment of the invention, insofar as the collection of acoustic information is concerned, a sensor with a somewhat dome-shaped configuration is provided which includes an acoustic chamber having a mouth spanned by a deflectable, preferably gas-permeable membrane, with an appropriate sound transducer, such as a microphone, an accelerometer, or a pressure sensor, coupled suitably to the acoustic chamber for the purpose of gathering heart-sound signals. The sensor is intended for direct, contactive placement at/adjacent a selected site on a subject's anatomy, is equipped with a very effective vacuumizing structure which utilizes less-than-atmospheric-pressure creation to produce a stable, contactive grip between the sensor and the anatomy, and utilizes, preferably, though not necessarily, direct compression contact between the membrane and the subject's anatomy at the selected site, whereby acoustic information of interest is very satisfactorily coupled through the membrane, into the acoustic chamber as an event, or as events, thence to be detected by the sound transducer.

The overall weight of the sensor of this invention is such that it possesses sufficient mass inertia to isolate, in a manner which will be explained, and for sound-information detection, actual heart-produced sounds transmitted through the membrane which, because of mass-inertia behavior, do not become minimized by otherwise possible, companion motion of the entire sensor in response to heart-sound pulsatile activity. Through utilizing what are referred to herein as skin-compression and skin-tension regions which become created when the sensor of this invention is applied to the anatomy, high-level electrical and acoustic coupling takes place for the accurate gathering of heart-activity information such as that mentioned above.

The various features and advantages that are offered and attained by the present invention will become more fully apparent as the description which now follows is read in conjunction with the accompanying drawings.

### DESCRIPTION OF THE DRAWINGS

FIGS. 1, 2 and 3 are photographic images from three different points of view illustrating a working model of the preferred embodiment of the sensor of the present invention.

FIGS. 5 and 6 are simplified, schematic, cross-sectional illustrations of the sensor pictured in FIG. 1-3, inclusive, with FIG. 4 illustrating the sensor in an isolated condition, and FIG. 5 illustrating the same sensor attached for use to a selected site in a human subject's anatomy. FIG. 5 also illustrates schematically herein what is referred to as an external spatial reference which is useful in the description of the methodologic operation of the present invention.

FIG. 6 is a very simplified and schematic illustration of what are referred to herein as regions of skin tension and skin compression.

### DETAILED DESCRIPTION OF THE INVENTION

Turning now to the drawings, indicated generally at 10 is an anatomy-contact sensor which is constructed, and which operates, in accordance with a preferred implementation of the present invention. Sensor 10 includes a generally dome-shaped body 12 which includes an axis of revolution 12a. Specifically forming body 12 herein are two differently sized, nested, outer and inner, dome-shaped portions 12b, 12c respectively, which are spaced from one another so as to define between them a generally dome-shaped clearance space 14 which is referred to herein as being part of a fluid-passage structure. Structure 14 includes a central port 14a which, as can be seen especially in FIGS. 4 and 5, communicates both with space 14, and through outer body portion 12b with an outwardly exposed port 16 which is referred to herein as an operational port. Ports 14a, 16 also form part of the just-mentioned fluid-passage structure. Structure 14 also includes an annular (annulus) aperture 14b which is referred to herein as a vacuumizing aperture. Aperture 14b, which faces generally downwardly in FIGS. 4 and 5, circumsurrounds, and is substantially centered on, axis 12a.

Body 12 herein is made of a suitable material, such as a suitable metal material, which, at least in the region of outer body portion 12b which is adjacent aperture 14b, is electrically conductive. As will be seen, the lower, annular rim of body portion 12b contacts a subject's anatomy to collect ECG electrical signals.

Inner body portion 12c, which is linked to outer body portion 12b in any suitable internal fashion, defines what is referred to herein as an acoustic chamber 18 possessing a generally circular mouth 18a (diameter herein about ⅞-inches) which is spanned by a thin, deflectible membrane 20. Membrane 20 effectively seals chamber 18 at the location of mouth 18a against the through-passage into chamber 18 of everything but gas which is flow-permitted as a consequence of membrane 20 preferably having the characteristic of gas permeability. A suitable material for forming membrane 20 is a product sold under the trademark Tyvek® made by DuPont—a material which possesses the just-mentioned gas permeability. This Tyvek® product is made of very fine, high-density polyethylene fibres, and exhibits properties of strength, tear and abrasion resistance, puncture resistance, low-linting tendency, and vapor-(but not liquid)permeability. Those skilled in the art will recognize that there are various, conventional and readily available, thin, membrane-effective materials which exhibit appropriate, preferred gas permeability. Any of these materials may readily be employed as a membrane in the practice of the invention.

Membrane 20 in sensor 10 furnishes what is termed herein as a gas-permeable region which is associated with chamber 18. Such a region allows for desirable fluid-pressure balance between chamber 18 and the outside environment. Such a gas-permeable region, however, may, if desired, be provided in other ways than by membrane 20. One of these ways could include an appropriately located, small, open vent which communicates the interior of chamber 18 with the “outside world”.

Attached to port 16, through a fitting portion 22a, is a pliable, hollow, squeeze bulb, or fluid-pumping structure, 22, which, together with the mentioned fluid-passage structure, is referred to herein collectively as vacuumizing structure. Those skilled in the art will recognize that something other than a squeeze bulb could be used to perform the function of this bulb.

Completing a description of the structure of sensor 10, communicatively coupled in any suitable fashion to the inside of chamber 18 is an acoustic(sound)-to-electrical-signal transducer 24 (see FIGS. 4 and 5) which may be, preferably, either a microphone, an accelerometer, or an appropriate pressure sensor. This transducer is shown anchored in an appropriate clearance space 12d (see FIG. 4) between body portions 12b, 12c in a condition where the transducer is disposed laterally to one side of body axis 12a. Not specifically illustrated in detail herein, but nonetheless to be understood as a possible modification, is that a transducer, such as transducer 24, may also be disposed, if desired, on axis 12a. The transducer may also be arranged to be in direct contact with membrane 20 in certain applications.

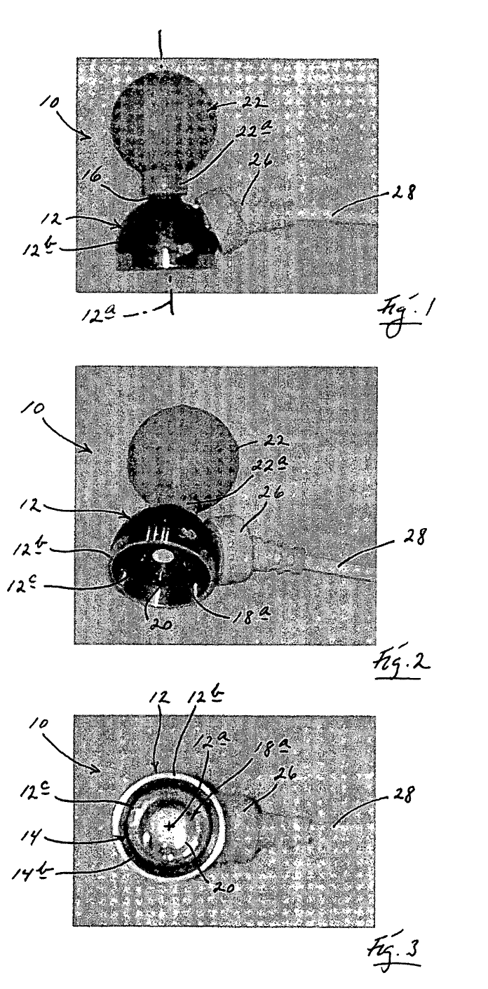

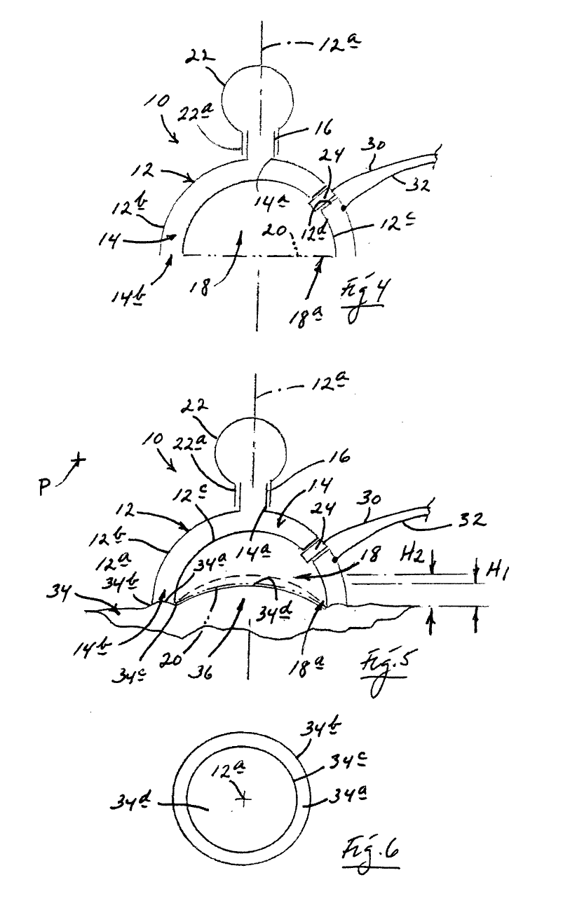

Extending outwardly away from sensor body twelve, through a strain-relief fitting 26 (see FIGS. 1 and 2), is a cable 28 which carries signal lines 30, 32 (see FIGS. 4 and 5). Line 30 carries acoustically-based electrical signals, and line 32 ECG electrical signals, to appropriate external structure (not shown). Line 30 connects with transducer 24, and line 32 with an electrically conductive part of outer body portion 12b.

The overall sensor structure has what is referred to herein as a mass M which, in the embodiment now being described, is about 28-grams. A suitable mass weight range for this sensor structure has been found to be about 10- to about 40-grams.

Describing now the operation of sensor 10 in accordance with practice of this invention, the lower side of sensor 10, as such is illustrated in FIGS. 5 and 6, is placed against (drawn adjacent) the anatomy at a selected site, with bulb 22 then squeezed and released to create a vacuum condition in the fluid passage structure, which vacuum condition is communicated to a subject's skin at the chosen anatomical site through annular aperture 14b. This vacuum condition causes the sensor to grip the skin surface at the selected anatomical site. In FIG. 5, a fragment of human anatomy is shown at 34, and the mentioned selected site is illustrated at 36.

This negative-pressure vacuum condition, which herein is about minus 5-lbs/in², lies preferably in the range of about minus 2-lbs/in² to about minus 15-lbs/in². A consequence of this vacuum condition is that the anatomy which is exposed to aperture 14b is placed in a condition of skin tension, whereby it bulges very slightly into the aperture, as can be seen at 34a in FIG. 5. Under this condition, the outer and inner body portions 12b, 12c, respectively, are pressed downwardly into the subject's skin to create regions 34b, 34c, respectively, of skin-compression, with a skin-compression bulge 34d occurring against contacting membrane 20, as can be seen in FIG. 5. Bulge 34d nominally has a height which is indicated in FIG. 5 by a pair of arrows marked H₁. In general terms, the size of bulge 34d is directly related to the magnitude of the created, negative-pressure vacuum condition.

Focusing attention for a moment on schematic FIG. 6, one can here see, from another point of view, the positional relationships of skin-compression regions 34b, 34c, 34d, and region of skin tension 34a, with sensor 10 in place as shown in FIG. 5. Accordingly, one can see that skin-tension region 34a circumsurrounds skin-compression regions 34c, 34d and that skin-compression region 34b circumsurrounds skin-tension region 34a. Skin-tension region 34a is thus bracketed by skin-compression regions 34b, 34c.

The vacuum condition just described, aided by the substantial mass M of the overall sensor structure mentioned above, helps to stabilize the sensor in place on the anatomy. Very specifically, the relatively large mass of the overall sensor structure tends, through inertia, to resist any tendency of the sensor structure as a whole to move as a consequence of acoustic pulsatile motion which takes place at anatomy site 36. A consequence of this important consideration is that acoustic-activity motion tends to produce good relative deflection of membrane 20, as illustrated very schematically by arrows H₂ in FIG. 5.

With respect to this last mentioned operating condition of the sensor of this invention, one can see that, by so utilizing inertial mass of sensor 10, relative motion which takes place between membrane 20 and any appropriate, external, fixed, spatial reference point, such as reference point P in FIG. 5, is greater than relative motion which takes place between the sensor structure as a whole and reference point P. Thus, the design of sensor 10, according to the invention, enhances the ability of transducer 24 correctly to collect, in a distinguishable fashion, heart-sound information, such as from heart sounds S1, S2, S3 and S4, which is communicated into chamber 18.

There are several different ways in which the methodology of this invention can be described. In one such way of description, this methodology can be seen to take the form of anatomy-contact, heart-activity sensing employing the steps of (a) utilizing a vacuum condition, drawing a gas-permeable-membrane-sided acoustic chamber against, or adjacent, the anatomy of a subject, and (b), following that drawing activity, collecting from the acoustic chamber acoustic-energy, anatomy-generated information based upon anatomy-induced acoustic events that occur in the acoustic chamber.

Another way of describing the behavior of the present invention is to characterize it as including the steps of (a) drawing an acoustic-activity-sensing, signal-generating sensor against (or adjacent), and in gripping contact with, a subject's anatomy, where the sensor possesses and utilizes a deflectible, anatomy-associating membrane directly to collect anatomical acoustic information, and (b), utilizing the phenomenon of mass inertia in the sensor, creating a condition wherein anatomical, acoustical motion behavior, relative to a fixed external spatial reference, produces greater relative motion between the membrane and the reference than between the sensor as a whole and the reference.

Yet another way of describing the methodology of this invention is to view it as a method for collecting heart-produced information including the steps of (a) creating, at a selected site in the anatomy of a subject, a pair of adjacent regions including a region of skin tension and a region of skin compression, (b) utilizing a skin-contact sensor, gathering heart-produced acoustic information from the region of skin compression, and (c), while so gathering acoustic information, utilizing the region of skin tension as an aid to hold the sensor in contact with the subject's anatomy.

Still a further methodologic description of the invention views it as the practice of collecting heart-produced information including the steps of (a) creating, at a selected site in the anatomy of a subject, a region of skin tension bracketed on opposite sides by a pair of adjacent regions of skin compression, (b) utilizing a skin-contact sensor, gathering heart-produced acoustic information from one of the created regions of the skin compression, and gathering heart-produced electrical information from the other region of skin compression, and (c), while so gathering information, utilizing the region of skin tension as an aid to hold the sensor in contact with the subject's anatomy.

In the practice of the invention, the intentional, elevated mass of the overall sensor causes motion of the sensor to be very slight in the presence of anatomical acoustic behavior, while motion of the membrane in response to that behavior is not so constrained. Cooperating with this special use of inertial mass to maximize relative membrane motion is the companion use of (a) a vacuum condition to draw the sensor tightly against the anatomy, whereby compression contact between the membrane and the anatomy becomes enhanced for improved coupling of acoustic information, and (b) enhanced compressive contact between the sensor body and the anatomy for improved ECG electrical-signal-gathering contact.

It should be evident to those generally skilled in the relevant art that the sensor and practice of the present invention, which offers superior collection of heart sound and electrical signals, can be subjected to a number of different modifications designed to suit particular applications. For example, the size and weight of the overall sensor structure can be varied, as can also be varied the type and location of an acoustic transducer. The sensor may be designed to create different vacuum-condition levels of less-than-atmospheric-pressure—conditions to be exposed to the anatomy at the location of aperture 14b. The structure employed to produce the less-than-atmospheric-pressure condition may also take different forms, and various other modifications may be made to suit particular use situations. The sensor structure may be made to be a patient-specific, discardable structure, or it may be made to be, in whole or in part, a reusable structure.

Accordingly, while a preferred embodiment, and manner of practicing, the invention have been described and illustrated herein, it is appreciated that variations and modifications may be made without departing from the spirit of the invention.

1. An anatomy-contact sensor for collecting at least heart-sound data comprising
a sensor body with an internal acoustic chamber having a mouth placeable adjacent a subject's anatomy,
a deflectible, anatomy-associating membrane spanning said mouth, and effectively sealing said chamber against through-passage in the mouth of all essentially but gas,
an acoustic-to-electrical-signal transducer operatively associated with said body and exposed to acoustic events occurring in said chamber, operable, on the occurrence of such events, to produce related electrical output signals, and
vacuumizing structure including (a) a vacuumizing aperture formed in said body adjacent and outwardly of said membrane, and (b) fluid-passage structure communicatively connected to said aperture, operable to enable the creation, through said aperture, of a releasable vacuum condition effective to hold said body in gripping contact with a subject's anatomy.

2. The sensor of claim 1 which includes a gas-permeable region associated with access to said chamber.

3. The sensor of claim 1, wherein said gas-permeable region is provided by said membrane.

4. The sensor of claim 1, wherein said membrane is disposed in the sensor to function as an anatomy-contacting structure.

5. The sensor of claim 1, wherein said transducer is at least one of (a) a microphone, (b) an accelerometer, and (c) a pressure sensor.

6. The sensor of claim 1, wherein said body has an axis of revolution, and said transducer is disposed on said axis.

7. The sensor of claim 1, wherein said body has an axis of revolution, and said transducer is disposed laterally to one side of said axis.

8. The sensor of claim 1, wherein said fluid-passage structure includes an operational port, and said vacuumizing structure further includes fluid-pumping structure operatively connected to said port.

9. The sensor of claim 1, wherein said fluid-passage structure includes an operational port, and said vacuumizing structure further includes a manipulable, vacuum-implementing, squeeze bulb attached to said port.

10. The sensor of claim 1, wherein said body has an axis of revolution, and said aperture takes the form of an annulus substantially centered on said axis.

11. The sensor of claim 1, wherein the overall structure of said sensor possesses a mass M having an inertia which, with the sensor in an operative condition in contact with a subject's anatomy, causes such overall structure to resist motion as a unit as urged by, and in response to, anatomical, acoustic, pulsatile movement-activity of the contacted anatomy, thus to enhance related, detectible, acoustic, motion-activity events produced by the anatomy in said chamber though anatomical contact with said membrane.

12. The sensor of claim 11, wherein said vacuumizing structure is constructed to produce a vacuum condition in said fluid-passage structure in a pressure range of about minus 2-lbs/in² to about minus 15-lbs/in² with said aperture in contact with a subject's anatomy, and said mass M has a weight lying in the range of about 10-grams to about 40-grams.

13. The sensor of claim 1 which further comprises at least one ECG electrode disposed on said body.

14. The sensor of claim 13, wherein said body has an axis of revolution, and said electrode takes the form of an annulus substantially centered on said axis.

15. Anatomy-contact, heart-activity sensing comprising
utilizing a vacuum condition, drawing a gas-permeable-membrane-sided acoustic chamber adjacent the anatomy of a subject, and
following said drawing, collecting from the chamber anatomy-generated acoustic information which is based upon anatomy-induced acoustic events occurring in the chamber.

16. The sensing of claim 15 which further comprises utilizing anatomy-contacting mass inertia to enhance the detectibility of acoustic-event information which is collectable from the chamber.

17. Anatomy-contact, heart-sound sensing comprising
drawing an acoustic-activity-sensing, signal-generating sensor against, and in gripping contact with, a subject's anatomy, where the sensor possesses and utilizes a deflectible, anatomy-contacting membrane directly to collect anatomical acoustic information, and
utilizing the phenomenon of mass inertia in the sensor, creating a condition wherein anatomical, acoustical motion behavior, relative to a fixed external spatial reference, produces greater relative motion between the membrane and the reference than between the sensor as a whole and the reference.

18. The sensing of claim 17, wherein said drawing is performed by implementing a less-than-atmospheric-pressure vacuum condition which is created in contact with a subject's anatomy, said drawing produces a compression bulge of anatomy against the membrane, and the size of the bulge and the associated compression are directly related to the magnitude of the less-than-atmospheric pressure existing in the vacuum condition.

19. A method for collecting heart-produced information comprising
creating, at a selected site in the anatomy of a subject, a pair of adjacent regions including a region of skin tension and a region of skin compression,
utilizing a skin-contact sensor, gathering heart-produced acoustic information from the region of skin compression, and
while so gathering, utilizing the region of skin tension as an aid to hold the sensor in contact with the subject's anatomy.

20. The method of claim 19, wherein said creating results in the region of skin tension circumsurrounding the region of skin compression.

21. The method of claim 19, wherein said creating of a region of skin tension is performed by employing a vacuum condition.

22. A method for collecting heart-produced information comprising
creating, at a selected site in the anatomy of a subject, a region of skin tension bracketed on opposite sides by a pair of adjacent regions of skin compression,
utilizing a skin-contact sensor, (a) gathering heart-produced acoustic information from one of the created regions of skin compression, and (b) gathering heart-produced electrical information from the other region of skin compression, and
while so gathering, utilizing the region of skin tension as an aid to hold the sensor in contact with the subject's anatomy.

23. The method of claim 22, wherein the mentioned one region of skin compression is circumsurrounded by the region of skin tension, and region of skin tension is circumsurrounded by the other region of skin compression.

24. The method of claim 22, wherein said creating of a region of skin tension is performed by employing a vacuum condition.
